(19) United States
(12) Patent Application Publication
(10) Pub. No.: US 2002/0098531 A1
(43) Pub. Date: Jul. 25, 2002

(54) Rapid methods for microbial typing and enumeration

(76) Inventor: James D. Thacker, Marietta, OH (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 10/053,871

(22) Filed: Jan. 24, 2002

Related U.S. Application Data
(60) Provisional application No. 60/263,761, filed on Jan. 25, 2001.

(51) Int. Cl. C12Q 1/70; G01N 33/554; G01N 33/569

(52) U.S. Cl. 435/7.32; 435/5

(57) ABSTRACT

The invention relates to kits and methods for the sensitive and rapid typing and enumeration of microorganisms in a sample. The basic method comprises adhering specific capture-antibodies to a solid support, to bind to microorganisms specific for the antibody, and adding primary antibodies specific to a viability marker of the microorganisms. This is followed by the addition of secondary antibodies that may be conjugated to a reporter molecule. Preferably the reporter function involves light and the detectable marker is aequorin and the need for a second antibody is overcome. The invention is useful for the detection of a number of different microorganisms including bacteria, fungi and protozoan of a variety of species.

### FIELD OF THE INVENTION

The present invention relates to kits and methods for the rapid typing and enumeration of microbial organisms. In particular, the invention involves the rapid and sensitive detection of microorganisms, especially bacteria, using antibody based capture assays in the clinical, pharmaceutical, environmental, cosmetic and water purification industries.

### BACKGROUND OF THE INVENTION

Microbial contamination has serious consequences, not only for its direct effect on health and health care, but also for its far reaching economic consequences. Bacteria, viruses, fungi, yeast and protozoans are responsible for an enormous number of diseases. While some of these diseases result from direct infection from a limited reservoir of pathogens, a great many are contagious allowing their spread from a limited reservoir to a greater population. Thus, infection from a small reservoir is capable of reaching epidemic proportions.

Microorganisms also pose a risk to non-human hosts. For example, some microbes that may not infect humans may be highly contagious to animals and livestock (e.g. foot and mouth disease, swine fever, bovine tuberculosis). Other microbes may pose a serious risk to plants, including crops such as cereals and grains, or even forests (such as Dutch Elm Disease, or Chestnut Blight). In addition, some pathogens, which have no clinical effect on their endogenous host, may cross the species barrier and have devastating effects on a naive host (including Ebola, Dengue Fever, Malaria and Avian Encephalitis to name a few). Further, some pathogens including E. coli and Salmonella are particularly pervasive in certain industrial applications such a meat packing, water treatment, and food production.

While the economic effect of non-fatal microbial contamination may be huge, the effect of contagious microbes can be devastating to enormous numbers of individuals. Diseases such as toxic shock, Legionnaires disease or Lyme disease have been lethal or result in serious health problems to large numbers of individuals in rich countries. However, the cost to poor countries is incalculable when wide-spread epidemics of diseases such as tuberculosis, cholera or influenza occur. The potential economic loss to the U.S. gross domestic product, alone, due to microbial contamination has been estimated to be $1-2 trillion (THACO Corporation, Independent Market Research, 1993).

In addition to the harmful effects of microbial contamination, there are also practical uses for microbes. A growing number of environmentally friendly methods for recycling waste and reclaiming toxic sites call for the inoculation of the target sites with specific percentages of microbes, including bacteria and fungi, that are capable of breaking down toxic substances, particularly when grown in synergy with each other. Thus, the relative concentrations of the mixed inoculum must be monitored on a periodic basis, sometimes in field conditions.

As is apparent from the foregoing, there are at least three principal reasons for monitoring the microbial concentration in a sample. The first is to determine whether any microorganisms are present; the second is to determine the microbial concentration if they are present; and the third is to determine the particular species of microbes in the sample.

Classically, the approach to answering these questions involves culturing the sample in the presence of selective nutrients and examining the sample microscopically after staining with specific reagents. While the classical approach can identify most organisms, its utility is based on the availability of time necessary to culture the organisms, on the skill of the microscopists in using techniques necessary to identify diverse organisms and in their competence to then make a correct determination.

Modem techniques for microbial identification and enumeration have focused on the development of more sensitive methods of detecting microorganisms and to a lesser extent upon improved methods for the amplification of the number of microorganisms present in the sample to be analyzed. These include the use of new techniques in molecular biology and biochemistry such as the use of DNA probes, RNA probes, ATP measurements, immunoassays, enzymatic assays and respirometric measurement. Many of these tests do not rapidly detect less than 10⁵ colony forming units per milliliter (cfu/ml) and still require complicated or lengthy amplification procedures to increase the concentration of the substrate being detected. In addition, these assays must be performed under highly controlled conditions and require skilled technicians to perform and interpret the results. Other strategies include the enhancement of the sensitivity of the detection system to reduce the threshold concentration of microorganisms needed for detection and consequently reduce the time required for amplification. These enhanced assay methods include fluorometric, radiometric and photometric methods. However, all these methods have their limitations.

Schapp (U.S. Pat. No. 4,857,652) identified compounds that can be triggered by an activating agent to produce light. This luminescent reaction is used for ultra sensitive detection of phosphatase-linked antibodies and DNA probes. At least one such application of this technology has been commercialized as Photo Gene™ manufactured by Life Technologies, Inc. (Gaithersburg, Md.). Similarly, Abbas and Eden ( U.S. Pat. No. 5,223,402) identify a method that uses 1,2-dioxetane chemiluminescent substrates linked to either alkaline phosphatase or β-D-galactosidase. Theoretically, their method can detect microorganism concentrations as low as 1-100 cfu/ml.

Although applicable in certain limited laboratory settings, these methods have several deficiencies. Chemiluminescent methods such as those described are susceptible to interference from a variety of chemical quenching agents commonly found in industrial waste waters, environmental water sources and biological matrices. Moreover, the methods as taught in the above-referenced patents require specialized equipment, multiple steps in the conduct of the assay and enrichment of the microorganism concentration. Taken together, such considerations lengthen the total assay time, raise the capital costs and make this technology unsuitable for high volume, high throughput applications.

Another strategy for the enhancement of microbial detection is the utilization of fluorescence based detection systems. For example, Fleminger (Eur. J. Biochem. 125:609-15, 1982) used a fluorescent amino benzoyl group that was intra molecularly quenched by a nitrophenylalanyl group. In the presence of bacterial aminopeptidase P, the nitrophenylalanyl group is cleaved and the fluorescence of the sample increased proportionately. A wide variety of other enzymes have been assayed by similar procedures and include hydrolases, carboxypeptidases and endopeptidases.

As is the case with the chemiluminescence based assays, fluorescence based assays also have severe limitations. Many fluorescence assays are susceptible to interference from chemical quenching agents typical in industrial processes and require specialized equipment and operator processing. In addition, some reagents such as those used in fluorescence, may be highly toxic and therefore not suitable for some applications. Further, while these methods may be amenable to the determination of the presence of particular microbes, they cannot discriminate between those microbes with a high degree of specificity.

Species typing, determining the particular species of a microorganism, is even more difficult in a complex sample. Species typing not only requires amplification of the microorganisms present, but also the selective detection of only those species of interest in the presence of background microflora. The classic approach to species typing is to selectively amplify the presence of the organism of interest through a pre-enrichment step followed by a selective enrichment step using a nutrient-specific media followed by biochemical or serological confirmation. The time required for these procedures can be as long as six to seven days which is clearly outside the realm of practicality for use in industrial laboratories or high throughput clinical laboratories.

One strategy that has recently been commercialized is the GENE-TRAK™ colorimetric assay (GENE-TRAK Systems, Inc. Framingham, Mass.). This technology attempts to simultaneously exploit an amplification strategy and an enhancement of the detection system's sensitivity. The approach is an alternative to other strategies that use probes directed against chromosomal DNA. Instead, the GENE-TRAK™ system targets ribosomal RNA (rRNA) which is present in 1,000-10,000 copies per actively metabolizing cell. A unique homologous series of nucleotides, approximately 30 nucleotides in length and containing a poly-dA tail, is hybridized with the unique rRNA sequence in the target organism. This probe is referred to as the capture probe. A second unique probe of 35-40 nucleotides is labeled at the 3′ and the 5′ ends with fluorescein. This probe is the detector probe and binds to a region of the rRNA adjacent to the capture probe. After hybridization, bound complexes are captured on a solid support coated with poly-dT, which hybridizes with the poly-dA tail of the capture probe. The rRNA-detector probe complex is detected with polyclonal anti-fluorescein antibody conjugated to horseradish peroxidase. This complex is then reacted with the enzyme substrate, hydrogen peroxide, in the presence of tetramethylbenzidine. The blue color that develops is proportional to the amount of rRNA captured. While this strategy is sensitive, RNA is a highly unstable molecule and any method utilizing it must be performed under strictly controlled conditions.

Blackburn reviewed the development of rapid alternative methods for microorganism typing as it pertains to the food industry (C de W. Blackburn, “Rapid and alternative methods for the detection of salmonellas in foods,” Journal of Applied Bacteriology, 75:199-214, 1993). Therein, Blackburn describes several techniques for detection of Salmonella that rely upon a selective pre-enrichment and enrichment approach to amplification, the best of which still required approximately six hours before detectable levels of Salmonella were present.

Blackburn also reviewed enhanced detection methods including measurements of metabolism, immunoassays, fluorescent-antibody staining, enzyme immunoassay, immunosensors, bacteriophages and geneprobes. Analysis times could be reduced to as short as 20 minutes; the detection limits were about 10⁵ cfu (Blackburn et al., “Separation and detection methods for salmonellas using immunomagnetic particles,” Biofouling 5:143-156, 1991). Similarly the detection limits could be reduced to as low as 1-10 cfu, however the enrichment protocols required 18-36 hours. In all cases, the described methods provided detection limits that were either too high or analysis times that were too long to be practical for application to industrial processes and high volume, high throughput clinical situations.

There have been numerous approaches to microorganism detection and typing. U.S. Pat. No. 4,376,110 (David et al.) relates to a solid-phase immunoassay employing a monoclonal capture antibody and a labeled secondary antibody. Alternatively, U.S. Pat. No. 4,514,508 (Hirshfeld et al.) relates to labeled complement and U.S. Pat. No. 4,281,061 (Zuk et al.); U.S. Pat. No. 4,659,678 (Forrest et al.); and U.S. Pat. No. 4,547,466 (Turanchik et al.) relate to other immunochemical variants. All of these methods require from 10³ to 10⁷ cfu/ml to reliably detect the target microorganisms. Necessarily, additional enrichment steps are required which add several hours to days to the assay procedure.

Various enrichment techniques and procedures are also important in any assay. For example, Valkirs (U.S. Pat. No. 4,727,019) and Hay-Kaufman (U.S. Pat. No. 4,818,677) relate to flow-through devices to capture cells and in situ immunoassay to detect the presence of the target organism. Schick (U.S. Pat. No. 4,254,082) relates to an ion exchange particle system for capturing the target organism and Chau (U.S. Pat. No. 4,320,087) relates to an activated charcoal coated bead capture device. All of these devices suffer several limitations such as small volume capacities, fouling from the presence of particulates in the sample or nonspecificity of the capture process. Consequently, these inventions are unsatisfactory for large volume, high throughput industrial and clinical applications.

As the preceding discussion shows, there has been much research into methods to assay for the enumeration and type of microorganism in a variety of samples. However, it is clear that there continues to be a need for the development of simple, sensitive, rapid, inexpensive and reliable detection systems with applicability to a broad scope of industrial, clinical and agricultural process requirements.

### SUMMARY OF THE INVENTION

While the inventions described above have attempted to rectify the failings of classical methods to quantify and type bacteria, limitations of the described methods still include the time necessary to culture microbial organisms, the lack of sensitivity of current detection methods and the need for controlled environment and well-trained technicians to perform the tests. It has been surprising discovered that the methods of the invention solve these problems and are also rapid, sensitive, easy to use and accurate.

The present invention provides for capturing specific microorganisms on a solid support, labeling those organisms with a viability substrate to produce a viability marker, digesting the cells, contacting the cellular debris with a primary antibody to the viability marker and contacting the primary antibody with a secondary antibody prepared to the primary antibody and conjugated to a reporter molecule. The reporter molecule is ready for detection in a sensitive and quantifiable manner.

In some embodiments of the present invention, capture antibodies to specific microbes are immobilized on a solid support such as the wells of a microtiter plate, test tube or any other suitable support serving to immobilize specific antigens. The capture antibodies are blocked with a non-specific protein, such as bovine serum albumin in PBS, and an aqueous sample contacted with the solid support/capture antibody complex. The sample does not need to be purified and may comprise a clinical sample, a food sample, a cosmetic sample, a pharmaceutical sample, an industrial sample, an environmental sample, a blood sample, a tissue sample, a tissue homogenate sample, a bodily fluid sample or any other such sample which may be contaminated by microbes.

After the sample is incubated with the immobilized capture antibodies, a viability substrate is added to the sample such that any actively respiring organisms will take up the substrate and convert it into a viability marker, which is a water insoluble molecule. After appropriate incubation the sample is aspirated and the well is rinsed of non-bound residue. The cells immobilized on the solid support are then digested (e.g. with enzymes or chemicals) exposing the intracellular contents. A primary antibody specific to the viability marker is added to the complex on the solid support, incubated for an appropriate amount of time, aspirated and the complex again washed of non-specific binders. A secondary antibody prepared against the primary antibody and conjugated to a reporter molecule is then contacted with the complex and the non-specific binders washed off of the solid support. The resulting complex, formed from the antibody-microbe-viability marker-antibody-antibody conjugate, is available for the detection of the reporter molecule.

The present invention solves the problems discussed herein by only detecting actively respiring organisms. It was surprisingly discovered that by coating the solid support with specific capture antibodies, microorganisms can rapidly and specifically be typed with a high degree of accuracy. As described in U.S. patent application Ser. No. 09/148,491, which is specifically and entirely incorporated by reference, by adding a viability substrate to the sample many copies of the viability substrate are taken up by the microbes. The viability substrate is then metabolized by the microorganisms to a single water-insoluble marker molecule. The viability marker accumulates rapidly and in direct proportion to the number of microorganisms present in the sample. Upon digestion of the microbes multiple antigenic sites for the primary antibody are exposed and thus, amplifying the substrate available for labeling with the primary antibody.

Because the antibody antigen reaction is specific at the molecular level, the sensitivity of the detection is limited by the sensitivity of the reporter molecule and the detector. It was surprisingly found that specific amplification of the primary antibody using a secondary antibody specific for the primary antibody, coupled with the use of an appropriate reporter molecule, microbes can be detected at very low concentrations. In some embodiments, this allows the accurate detection of as little as 1 to 10 microbes.

In some embodiments of the present invention that the reporter molecule is a photoprotein; in particular the photoprotein may be a luminophor or a fluorophor. In other embodiments the reporter molecule is an enzyme, a radioisotope, a fluorescent dye, a chemiluminescent dye, a visible dye, a latex particle, a magnetic particle, a fluorescent dye or a combination thereof.

Those of skill in the art will recognize that other embodiments of the invention are possible. For example, the primary antibody may be directly conjugated to the reporter molecule, obviating the need for a secondary antibody. In these embodiments, as previously described, the sample plate is then read by the detector appropriate for the type of reporter molecule used.

As will be recognized by those of skill in the art, the present invention can readily be used as a pre-made kit where primary antibodies of any available specificity can be adhered to the solid support and kept in appropriate conditions to maintain the viability of the antibody. The kit includes all necessary reagents such as the wash solutions, primary and secondary antibodies and the trigger buffer or detection reagents. With these materials, the investigator may add a sample to all wells of the plate and determine the presence of any specific microbe with a high degree of accuracy both for quantity and type.

### DESCRIPTION OF THE DRAWINGS

FIG. 1 is a quantitative analysis of a mixed bacterial culture. This analysis was performed using classical methods of bacterial culture and microscopic identification.

FIG. 2 is a BactoType™ analysis of the mixed culture from FIG. 1. This analysis shows that the percentage of E. coli identified by the BactoType™ assay agrees with that calculated by the classical methods used in FIG. 1.

FIG. 3 shows the total viable bacteria as determined by the BactoLite™ assay. BactoLite™ assays of a pure culture of E. coli (●), H. influenzae (□) and a mixed culture (∘) from 10 cfu/ml to 10 million cfu/ml. Each data point is the average of duplicate measurements.

FIG. 4 shows the quantification of cell cultures with BactoType™ assays. Assays of pure cultures of E. coli (●) and H. influenzae (∘) with BactoType™ demonstrating linearity from 10 cfu/ml to 10 million cfu/ml.

FIG. 5 Represents an E. coli standard curve. The correlation coefficient (R²) of the best fit linear regression and the corresponding equation of the line are shown.

### DESCRIPTION OF THE INVENTION

As embodied and broadly described herein, the present invention is directed to kits and methods for the rapid typing and enumeration of microorganisms including, but not limited to, bacteria, fungi and protozoans. As described in the following embodiment, and will be clear to those skilled in the art, the present invention may also be used as a method for detecting the presence of bacteria including pathogenic bacteria in clinical, environmental and food samples. As such, the disclosed invention is a valuable tool for the diagnosis of sub-clinical disease states, microscopic contamination of food and water samples, and provides an excellent tool with which to monitor the type and quantity of any species that might exist latently in an isolated reservoir. These methods may be used to specifically detect the presence of a discrete number of microbes to specifically determine and quantify the presence of one or many microorganisms comprising a variety of species or serotypes found in an aqueous sample for which antibodies are available. In some embodiments, the method is sensitive enough to detect less than 10 cfu/ml and even 1 cfu/ml. In other embodiments, the invention is sensitive enough to detect less than 100 cfu/ml. In yet other embodiments, the invention is sensitive enough to detect less than 500 cfu/ml, while in other embodiments the invention is sensitive enough to detect less than 1000 cfu/ml.

As used herein, the term “typing” refers to the specific determination of the genus and/or species and/or serotype of the microorganism. As disclosed by the present invention, microbes are “typed” by the ability of antibodies produced specifically to that microbe to capture the microorganism to the solid support. The captured microbes are then detected on the basis of the secondary antibody-reporter conjugate. To type a microbial organism, a solid support is used to which specific antibodies are immobilized. Solid supports may be composed of glass, plastic, PVC or any other appropriate material. Examples of solid supports, such as Corning Costar assay plates or tubes (Fisher Scientific; Pittsburgh, Pa.), Falcon plates or tubes (Becton-Dickinson; Franklin Lakes, N.J.) and Nunc OmniTray (Fisher Scientific; Pittsburgh, Pa.) are commercially available.

Antibodies may be obtained from a variety of sources and includes, but is not limited to, a molecule that contains a binding domain capable of binding to a specific antigenic epitope. In some embodiments, the antibody may be any member of the immunoglobulin superfamily, including IgD, IgE, IgG, and IgM, humanized versions of any type and fragments thereof, or monoclonal or polyclonal antibodies or fragments thereof. In other embodiments the antibody may constitute only the binding domains of the variable heavy and/or variable light chain complementary determining regions, including antigen binding fragments (Fab), single chain or double chain variable fragments (Fv) or any other domain capable of binding specific epitopes. Antibodies may be prepared from recombinant cells including recombinant hybridoma cells. Recombinant hybridoma cells expressing specific antibodies can be obtained; for example, from the American Type Culture Collection or a variety of commercial sources such as Becton-Dickinson (Franklin Lakes, N.J.), Fisher Scientific (Pittsburgh, Pa.), Stratagene (La Jolla, Calif.), MorphoSys (Martinsreid, Del.) or Cambrindge Antibody Technology (Cambridge, UK). Where recombinant cells are cultured the antisera are harvested and centrifuged to remove cellular debris, and purified by passage through Protein A. Optimum dilutions in 10 mM phosphate buffered saline, pH 7.2 (PBS) of the Protein A purified antisera to be used in the assay can be determined by a checkerboard assay with goat, anti-mouse IgG conjugated to alkaline phosphatase (Sigma Chemical Company) as the probe.

To prepare the solid support, the plates or tubes for use as binding substrates are coated with optimized dilutions of antibody for two hours or less, and preferably less than 30 minutes. The antibody may be immobilized on the support by covalent bonding, ionic bonding, electrostatic bonds, van der Waals forces, hydrogen bonds or any other method of immobilizing the antibody or antibody fragment. The antibody solution is then aspirated from the well and the well blocked with 1% bovine serum albumin in PBS to reduce non-specific binding. Samples are diluted to contain approximately 10⁷ viable cells/ml and then can be serially diluted in decade increments such that the final dilution has a concentration of approximately 10¹ cells/ml. By this method a plate will have dilutions of the sample correlating to the linear portion of a calibration curve. Two hundred microliters of each dilution is then added to each well and is allowed to incubate at room temperature with shaking for 30 minutes, preferably lees such as, for example, 15 minutes. After the sample is added to the solid support, a viability marker is added to the suspension. The viability marker is a microbial-enzyme substrate (viability substrate) which when incubated with the cells in the sample is taken up and may be metabolized by the actively respiring microorganisms and, for example, produce a metabolic product. The viability substrate is metabolized by the microorganisms to one or more marker molecules (e.g. metabolic products or by products of metabolism, which may be water soluble or insoluble depending on the method of detection). Viability marker accumulates rapidly and in direct proportion to the number of microorganisms present in the sample. In addition, viability marker may accumulate within the microorganism. In some embodiments the viability marker may accumulate within the organism up to 100 copies, in other embodiments, viability marker may accumulate up to 1,000 copies while in other embodiments, marker may accumulate up to 1,000,000 copies. Thus, a single microorganisms may have up to 1,000,000 copies of the marker intracellularly affording over 1,000,000 targets for labeling by the primary antibody.

After incubation, which may be from minutes to hours to days, and is preferably less than about twenty four hours, less than about eight hours, less than about two hours, and still more preferably less than about thirty minutes and less than about ten minutes, microorganisms are digested in a manner to produce cell fragments with the viability marker adsorbed to the surfaces of the cellular debris. Digestion of the microbes may be achieved by any appropriate method including, chemical, enzymatic or detergent methods such as cell lysis. In addition, lysis of the cells can occur due to osmotic gradients or mechanical means such as occurring in a French press. Primary antibodies specific to the viability marker are added to the sample and affinity adsorbed to the surface of the cellular debris. Secondary antibodies, specific to the primary antibody, are conjugated or otherwise associated to a detectable reporter molecule (e.g. enzyme, dye, fluorophor, luminescent protein, magnetic beads, radioisotope or any other suitable molecule or combination of molecules). The reporter molecule is then quantitatively detected either directly or indirectly by the appropriate detector, if necessary, after the addition of the appropriate activator or enzyme substrate.

In a preferred embodiment, reporter molecule is a luminescent protein such as aequorin conjugated to a goat anti-rabbit IgG (SeaLite Sciences, Inc., Norcross, Ga.; Chemicon, Int., Temecula, Calif.). The flash luminescence resulting from the automatic addition of 200 μL of a trigger buffer (containing Ca²⁺ for aequorin) lasts for approximately 10 seconds. Detection of the reporter molecule is made with the appropriate instrument. For example, when the reporter molecule is a luminescent protein a luminometer is used for detection. Flash luminescence readings can be taken with a variety of commercially available luminometers (for example the MLX Luminometer available from Dynex Technologies, Inc.; LB 96V PerkinElmer, Norwalk, Conn.; LUMIstar, BMG Labtechnologies Inc., Durham, N.C.). Recent advances in photometric technology have made the detection of small releases of light quantifiable if properly controlled. For example, modern spectrophotometers and luminometers have a high degree of automation so that important parameters are carried out entirely within the instrument, thereby keeping most variables constant. For example, the MLX Luminometer (Dynex Technologies, Chantilly, Va.) automatically calibrates itself, injects the appropriate amount of buffer triggering the luminescent flash and quantifies the light emitted before moving to the next sample well. In addition, this luminometer has a dynamic range of eight decades with a maximum sensitivity of 0.0001 Relative Light Units (RLU). The MLX Luminometer takes one reading every 10 milliseconds, or 100 readings per second. Consequently, the determination of the viability marker bound by the primary antibody-secondary antibody conjugate can be objectively determined by the instrument. In addition, while the examples herein disclosed use a 96 well microtiter plate, other variations may be used such as an 8 well plate, a 384 well plate, a 496 well plate or a rack assembly.

Fully automated luminometers and spectrophotometers robotically control many of the variables responsible for error in sensitive assays. For example, the MLX Luminometer adds appropriate volumes of trigger buffer, mixes the contents of the wells and the relative light units (RLU) are summed over a one second read time per well. The number of relative light units can then be correlated against a standard curve and the number of microorganisms can be determined. In some embodiments, the invention herein described may take less than 120 minutes to perform the analysis. In yet another embodiment the time for analysis is less than 60 minutes, preferably less than 30 minutes and more preferably less than 15 minutes.

Other embodiments may also be apparent to one of skill in the art. For instance the primary antibody can be conjugated to the reporter molecule and the capture antibody-sample complex detected by the primary antibody without the addition of a secondary antibody. In addition, the reporter molecule may include a variety of substances such as enzymes, dyes, latex particles, magnetic beads or any other substance suitable for detection. In another embodiment the microbes can be digested prior to their application to the capture antibody.

The invention is further described by the following examples which are illustrative of the invention but do not limit the scope of the invention in any manner.

### EXAMPLE 1

### Analysis of Mixed Bacterial Culture

Preparation of Cultures

Materials

Sterile, opaque white 96-well micro-plates were purchased from Corning Costar. Sterile Durapore™ (0.45 u) microfilter plates and the Multiscreen™ filtration manifold were purchased from Millipore Corporation. BactoLite™ Substrate Reagent, BactoLite™ Digestion Reagent, and the BactoLite™ Primary Antibody are as described in U.S. patent application Ser. No. 09/148,491 and PCT App. No. US98/18588. AquaLite® Secondary Antibody (SeaLite Sciences, Inc., Norcross, Ga.; Chemicon International, Temecula, Calif.) is an antiglobulin to the primary antibody and is conjugated to aequorin as a flash luminescence marker. BactoLite™ Dilution Buffer was prepared from 1% BSA in 25 mM Tris, 0.145 M NaCl, pH8. AquaLite® Wash Buffer was prepared from 20 mM Tris, 5 mM EDTA, 0.15 m NaCl, 0.05% Tween-20™, pH 7.5 containing 15 mM sodium azide. AquaLite® Trigger Buffer was prepared from 50 mM Tris, 10 mM calcium acetate, pH 7.5 containing 15 mM sodium azide. Flash luminescence readings were measured in Relative Light Units (RLU) using an MLX Microtiter plate luminometer from Dynex Technologies, Inc.

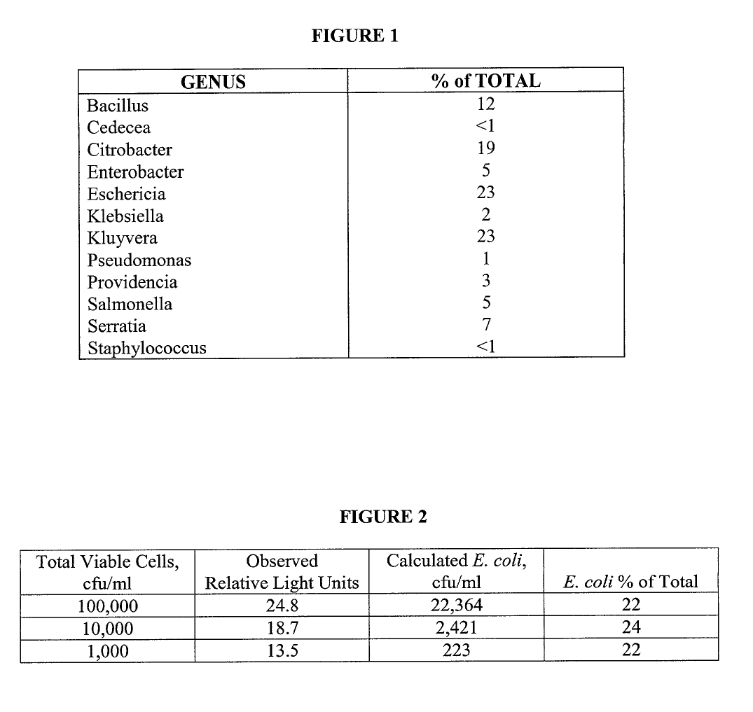

A mixed bacterial culture was isolated from pooled industrial cooling tower waters collected during the summer of 1993. One liter of the pooled water sample was filtered through a 0.2 um Durapore® membrane filter (Millipore Corporation) and the filter was placed into a culture flask containing 1 L of trypticase soy broth. The inoculated media was incubated aerobically at 37° C. with shaking on a rotating mixer set at nominally 80 rpm. Cells were harvested in mid-log growth phase by centrifugation. The cells were suspended in 50 ml of sterile trypticase soy broth and this suspension was further diluted 1:1 with sterile 20% glycerol in trypticase soy broth. The culture was distributed in 3 ml portions into sterile, screw-cap, amber vials. The culture, thus expanded and suspended, was stored frozen (−80° C.) at a cell density of 8.1×10⁹ cfu/ml. A quantitative analysis by genera for the mixed culture is presented in FIG. 1.

The following were obtained from the American Type Culture Collection: Escherichia coli ATCC 25922, and Haemophilus influenzae ATCC 49766. E. coli was grown in trypticase soy broth at 37° C. for 24 hours. H. influenzae was cultured on BBL® Chocolate II agar (Becton Dickinson) at 37° C. with 5% CO₂ for 48 hours. Cells were harvested from the plates using a sterile loop and resuspended in 5 ml of filter sterilized 0.85% NaCl for use in the subsequent assays. Serial ten-fold dilutions of the broth cultures or bacterial suspensions were made in 0.85% NaCl. A 100-μL aliquot was removed from the 10⁻⁵, 10⁻⁶ and 10⁻⁷ dilutions, spread plated on appropriate media and plates were incubated under the appropriate conditions. Total plate counts for each dilution were utilized to determine the standard cell counts (cfu/ml) to be used as a reference point in the BactoLite™ assay.

Preparation of Type Specific Microplates

Monoclonal antibodies for type specific antigens of E. coli (K99 pili) and H. influenzae (outer membrane protein P6) were purified from mouse hybridoma cell lines procured from the American Type Culture Collection (ATCC # HB-8178 and HB-9625 respectively). Hybridomas for E. coli were propagated in Dulbecco's modified Eagle's medium with 4.5 g/L glucose (85%) and fetal bovine serum (15%) and the hybridomas for H. influenzae were propagated in modified Dulbecco's medium (80%) and fetal bovine serum (20%). The antisera was harvested, centrifuged to remove cellular debris, and purified by passage through Protein A. No further purification was performed. Optimum dilutions in 10 mM phosphate buffered saline, pH 7.2 (PBS) of the Protein A purified antisera to be used in the assay were determined by a checkerboard assay with goat, anti-mouse IgG conjugated to alkaline phosphatase (Sigma Chemical Company) as the probe.

Using the Corning Costar microplates, wells of columns 1-4 were coated with goat, anti-rabbit IgG (Sigma Chemical Co.; St. Louis, Mo.) as negative controls. Columns 5-8 were coated with optimized dilutions of anti-E. coli while columns 9-12 were coated with optimized dilutions of anti-H. influenzae. All wells were blocked with 1% bovine serum albumin in PBS for approximately 30 minutes to reduce non-specific binding effects.

Conduct of the BactoLite™ Assay for Total Viable Cells

The mixed culture and pure cultures of E. coli and H. influenzae were analyzed according to the BactoLite™ method described by Thacker (Thacker, U.S. patent application Ser. No. 09/148,491; Thacker & George, 1988) to determine the total viable cell count. Wells in row A of the microfilter plates received sterile Dilution Buffer and served as background subtracted from the sample wells. Rows B & C contained decade serial dilutions of the mixed culture. Rows D & E contained decade serial dilutions of the E. coli culture. Rows F & G contained decade serial dilutions of the H. influenzae culture. Row H was unused. Duplicate measurements were averaged.

Actively respiring microorganisms were amplified by contacting the contents of the sample to a nutrient medium containing a predetermined amount of a viability substrate, wherein metabolism of the viability substrate by the microorganisms of said sample produces a viability marker. The viability substrate was a tetrazolium salt, which is metabolized by the microorganisms to produce a water insoluble marker molecule that accumulated in direct proportion to the number of microorganisms in the sample.

Tetrazolium salts that can be added to viable microorganisms to produce a detectable marker after metabolisms by the microorganisms include dimethylthiazolyldiphenyl tetrazolium, iodonitrotetrazolium, nitrotetrazolium blue or triphenyltetrazolium. The predetermined amount of tetrazolium salt is between about 0.01 mg/ml and 10.0 mg/ml, preferably from about 0.1 to about 1.0 mg/ml, and more preferably from about 0.2 to about 0.6 mg/ml. Viability substrates useful in the practice the invention may include any nutrient. In the preferred embodiment, the nutrient media is devoid of reducing sugars such as glucose to prevent non-specific reduction of the viability substrate. Where a nutrient media contains reducing sugars an excess of a mild oxidizing agent such as, for example, NAD⁺, NADP⁺, alpha keto acids, and many other known to those of ordinary skill, can be added to the nutrient media. As is clear to those skilled in the art, other nutrient sources such as other carbohydrates are well-known and can be used in addition to other known oxidizing agents.

Conduct of the BactoType™ Typing Assay

Using the type-specific microplates previously prepared, samples diluted to contain approximately 10⁷ viable cells/ml were serially diluted in seven decade increments and 200 μL of each dilution was applied to the wells as follows. The wells of columns 1,5 and 9 received sterile Dilution Buffer and were background subtracted from the sample wells. The wells of Columns 2, 6, and 10 received the eight dilutions of the mixed culture. Wells of columns 3, 7 and 11 received the dilutions of the E. coli culture while the wells of columns 4, 8 and 12 received the dilutions of the H. influenzae culture. After addition of the sample dilutions, the plate was incubated at room temperature with shaking for 15 minutes in the presence of the viability substrate. Samples were then aspirated and the wells washed 3× with wash buffer. The BactoLite™ digestion reagent was reconstituted with 25 ml of PBS and 200 μL was diluted to 20 ml in BactoLite™ assay buffer. Two hundred ml of the diluted primary antibody was added to each well of the solid support. The plate was incubated 30 minutes at room temperature with shaking on the orbital mixer, and the primary antibody removed by vacuum filtration. Each well was washed in the manner described above.

AquaLite® secondary antibody (goat, anti-rabbit IgG conjugated to aequorin, SeaLite Sciences, Inc., Norcross, Ga.; Chemicon International, Temecula, Calif.) was reconstituted in AquaLite® reconstitution buffer and diluted 1:100 in BactoLite™ Assay Buffer (25 mM Tris, 10 Mm EDTA, 2 mg/ml BSA 0.15 m KCl, 0.05% Tween-20, 15 mM sodium aide, pH 7.5) and 200 μL was added to each well of the microfilter plate. The plate was incubated 30 minutes at room temperature on a rotating mixer. After incubation the contents of the wells were removed by vacuum filtration and washed 3× with washing buffer as previously described.

Because the BactoType™ assay uses the power of the BactoLite™ system but begins with type specific capture antibodies immobilized on the solid support, each reading for the reporter molecule is specific for the microorganism captured by the capture antibody. Consequently, the power of the amplification system described in U.S. patent application Ser. No. 09/148,491 has surprisingly been harnessed to specifically type microbial species immobilized on the solid support by the capture antibody.

Flash Luminescence Readings

Flash luminescence readings were taken using an MLX Luminometer (Dynex Technologies, Inc.). The total integral of relative light units was summed over a one second read time per well after the automatic addition of 200 μL of AquaLite® Trigger Buffer (50 mM Tris, 10 mM calcium acetate, 15 mM sodium azide, pH 7.5). The microfilter plate was maintained at 35° C. during the data acquisition phase. The raw emission data was collected and processed by the luminometer and then down-loaded to a Microsoft Excel® spreadsheet for further analysis. Results are given in FIG. 2.

Determination of Total Culturable Bacteria

The standard plate count method was used to determine the total culturable bacteria in colony forming units per ml (cfu/ml) for each of the three test cultures. The results of the BactoLite™ assay in relative light units (RLU) were plotted against the log cfu/ml for each culture. These results are presented in FIG. 1. All three cultures showed a linear response to nominally 10 million cfu/ml. The E. coli response was linear down to nominally 10 cfu/ml representing approximately 2-5 viable bacterial cells per micro-well. The H. influenzae and mixed culture responses were linear down to nominally 100 cfu/ml representing 20-50 viable bacteria cells per micro-well.

Decade serial dilutions from 10 million cfu/ml to nominally 10 cfu/ml from all three of the cultures were analyzed on the BactoType™ plate prepared as described above. None of the cultures had a response above the background in the goat, anti-rabbit immunoglobulin coated (negative control) regions of the plate. The E. coli and the H. influenzae dilution series were detected in the corresponding anti-E. coli and anti-H. influenzae capture regions of the plate with no detectable cross reactivity above background. A plot of the E. coli and H. influenzae response (RLU v. log cfu/ml) is presented in FIG. 2. Both cultures reached a saturation end-point in the dose response after nominally 10,000 cfu/ml which was probably due to saturation of the immobilized capture antibodies. The E. coli culture showed a linear dose response range from nominally 10 cfu/ml to nominally 10,000 cfu/ml while the H. influenzae culture showed a linear dose response over the range from nominally 100 cfu/ml to nominally 10,000 cfu/ml.

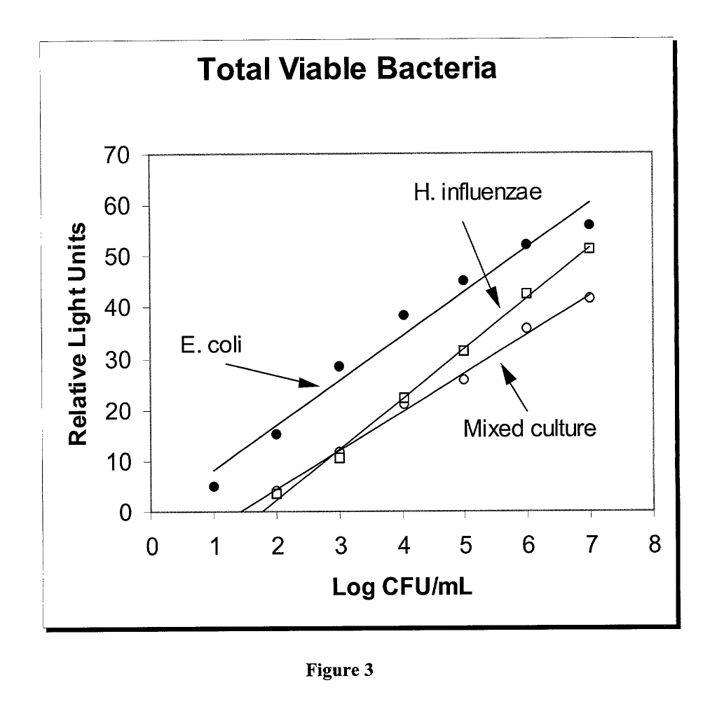

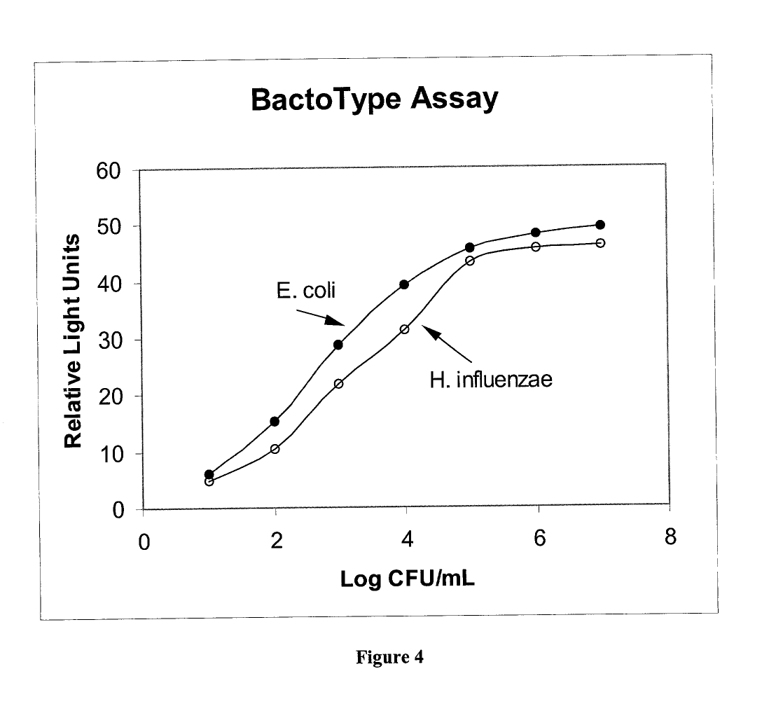

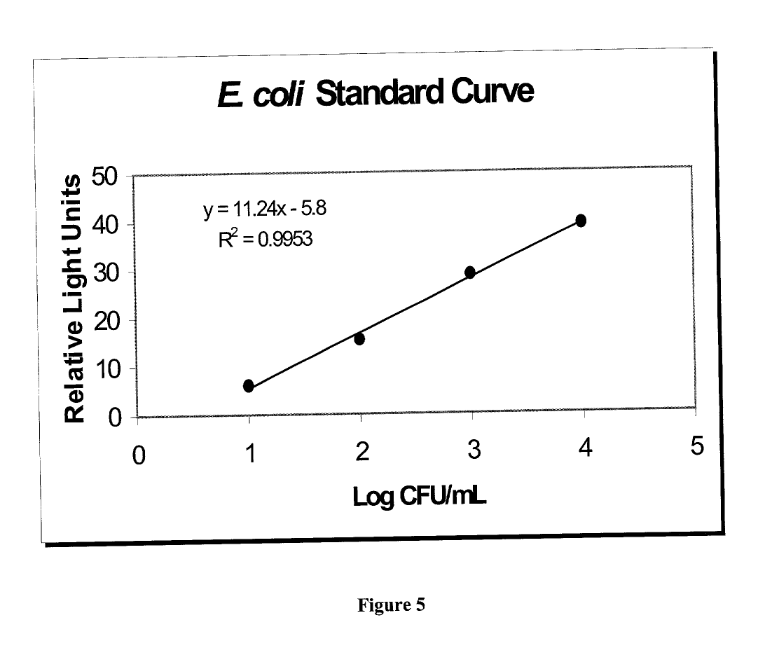

The mixed culture had no detectable response in the anti-H. influenzae capture region of the plate. This result is consistent with culture typing methods used to type and enumerate the various genera and species of bacteria present in the mixed culture (see FIG. 1). A dose response for the mixed culture in the E. coli capture region of the plate was observed from nominally 100 cfu/ml to nominally 10 million cfu/ml. To estimate the quantity of E. coli in the mixed culture, a standard curve using the linear region of the E. coli pure culture was established. FIG. 3 shows the standard curve, the correlation coefficient (R²) of the best fit linear regression line, and the corresponding equation of the line. The observed RLU at 1,000, 10,000, and 100,000 cfu/ml in the mixed culture were substituted into the equation for the regression line and the concentration of E. coli in the mixed culture was calculated by solving for “X”. The average percentage of E. coli calculated in the mixed culture was determined to be 23% which is the same value determined by the standard plate count methodology in FIG. 1. These results are presented in FIG. 2.

The results of the preceding experiments establish the exquisite sensitivity and linearity of the BactoType™ typing assay. Moreover, the BactoType™ assay as exemplified herein is highly sensitive and in some embodiments is capable of detecting microorganisms in less than one hour. As such, BactoType™ represents an enormous breakthrough methodology for rapid microbial typing. As exemplified herein, it is evident that so long as a capture antibody specific to an exposed protein of the microbe is immobilized on a solid support, virtually any bacterial species can be selectively detected. BactoType™ has diverse applicability to a wide variety of clinical and non-clinical applications including medical, environmental, food safety, animal health, public health, and industrial, markets.

Other embodiments and uses of the invention will be apparent to those skilled in the art from consideration of the specification and practice of the invention disclosed herein. All references cited herein for any reason, including all U.S. and foreign patents and patent applications, are specifically and entirely incorporated by reference. It is intended that the specification and examples be considered exemplary only, with the true scope and spirit of the invention indicated by the following claims.

1. A method for the rapid typing or enumeration of microorganisms comprising:
immobilizing a capture antibody on a solid support;
contacting a said immobilized capture antibody with a sample;
contacting the contents of said sample with a predetermined amount of substrate, wherein metabolism of said substrate by the microorganisms produces a marker;
digesting the microorganisms;
adding a primary antibody specific to said marker;
adding a second antibody specific for said primary antibody; and conjugated to a reporter molecule;
detecting the reporter molecule conjugated to the second antibody; and
determining the type or quantity of microorganism present.

2. The method of claim 1, wherein the digestion of said microorganisms comprises cell lysis.

3. The method of claim 1, which is capable of detecting 1000 colony forming units per ml or less of said microorganism.

4. The method of claim 1, which is capable of detecting 100 colony forming units per ml or less of said microorganism.

5. The method of claim 1, wherein the sensitivity of said method is capable of detecting 10 colony forming units per ml or less of said microorganism.

6. The method of claim 1, wherein the type or enumeration of microorganisms is determined in less than two hours.

7. The method of claim 1, wherein the type or enumeration of microorganisms is determined in less than one hour.

8. The method of claim 1, wherein the reporter molecule is selected from the group consisting of: a bioluminescent protein, a chemiluminescent dye, a fluorescent dye, an enzyme, a latex particle, a magnetic particle, a radioisotope, a visible dye, and combinations thereof.

9. The method of claim 1, wherein the substrate is dimethylthiazolyldiphenyl tetrazolium, iodonitrotetrazolium, nitrotetrazolium blue, or triphenyltetrazolium.

10. The method of claim 1, wherein the microorganism comprises one or more species of bacteria.

11. The method of claim 1, wherein the sample is selected from the group consisting of a bodily fluid, a blood sample, a clinical sample, a cosmetic sample, an environmental sample, a food sample, an industrial sample, pharmaceutical sample, a tissue sample, a tissue homogenate, and combinations thereof.

12. The method of claim 1, wherein the microorganisms are digested prior to their contact with said capture antibody.

13. A method for the rapid typing or enumeration of microorganisms comprising:
immobilizing a capture antibody on a solid support;
contacting a said immobilized capture antibody with a sample;
contacting the contents of said sample with a predetermined amount of substrate, wherein metabolism of said substrate by the microorganisms produces a marker;
digesting the microorganisms;
adding a primary antibody specific to said marker;
detecting said primary antibody bound to said marker; and
determining the type number of microorganisms present in said sample.

14. The method of claim 13, wherein the digestion of said microorganisms comprises cell lysis.

15. The method of claim 13, which is capable of detecting 1000 colony forming units or less of said microorganism.

16. The method of claim 13, which is capable of detecting 100 colony forming units or less of said microorganism.

17. The method of claim 13, wherein the sensitivity of said method is capable of detecting 10 colony forming units or less of said microorganism.

18. The method of claim 13, wherein the type or enumeration of microorganisms is determined in less than two hours.

19. The method of claim 13, wherein the type or enumeration of microorganisms is determined in less than one hour.

20. The method of claim 13, wherein the substrate is dimethylthiazolyldiphenyl tetrazolium, iodonitrotetrazolium, nitrotetrazolium blue, or triphenyltetrazolium.

21. The method of claim 13, wherein the microorganism is one or more species of bacteria.

22. The method of claim 13, wherein the sample is selected from the group consisting of a bodily fluid, a blood sample, a clinical sample, a cosmetic sample, an environmental sample, a food sample, an industrial sample, pharmaceutical sample, a tissue sample, a tissue homogenate, and combinations thereof.

23. The method of claim 13, wherein the microorganisms are digested prior to contact with the capture antibody.

24. The method of claim 13, wherein the primary antibody is conjugated to a reporter molecule.

25. The method of claim 24, wherein the reporter molecule is selected from the group consisting of: a bioluminescent protein, a chemiluminescent dye, a fluorescent dye, an enzyme, a latex particle, a magnetic particle, a radioisotope, a visible dye, and combinations thereof.

26. A kit for the rapid detection or enumeration of microscopic organisms comprising:
a solid support;
capture antibodies affixed to said solid support;
a soluble substrate which upon uptake by actively respiring organisms is metabolized to a water-insoluble molecule;
a primary antibody specific for said water-insoluble molecule; and
a second antibody specific for said primary antibody and conjugated to a reporter molecule.

27. The kit of claim 26, wherein the solid support is supplied with said capture antibodies immobilized thereto.

28. The kit of claim 26, further comprising a wash buffer, a dilution buffer, and a digestion reagent.

29. The kit of claim 26, wherein the reporter molecule is selected from the group consisting of a bioluminescent protein, a chemiluminescent dye, a fluorescent dye, an enzyme, a latex particle, a magnetic particle, a radioisotope, a visible dye, and combinations thereof.

30. The kit of claim 26, wherein said reporter molecule comprises an enzyme.

31. The kit of claim 26, further comprising a nutrient media.

32. The kit of claim 31 wherein the nutrient media comprises a reducing sugar and a mild oxidizing agent

33. The kit of claim 32 wherein the mild oxidizing agent is NAD⁺ and the reducing sugar is glucose.

34. A kit for the rapid detection or enumeration of microscopic organisms comprising:
a solid support;
capture antibodies affixed to said solid support;
a soluble substrate which upon uptake by actively respiring organisms is metabolized to a water-insoluble molecule; and
a primary antibody specific for said water-insoluble molecule.

35. The kit of claim 34, wherein the primary antibody is conjugated to a reporter molecule.
